(19) United States
(12) Patent Application Publication
(10) Pub. No.: US 2008/0262014 A1
(43) Pub. Date: Oct. 23, 2008

(54) Use of Camptothecin Derivatives for the Treatment of Proliferative Diseases in a Fixed Dosing Regimen

(76) Inventors: Horst F. Schran, Morristown, NJ (US); Margaret Ma Woo, Raritan, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/592,832

(22) PCT Filed: Mar. 24, 2005
(86) PCT No.: PCT/EP2005/003183

§ 371 Date: Jul. 7, 2008

Related U.S. Application Data
(60) Provisional application No. 60/556,729, filed on Mar. 26, 2004.

(51) Int. Cl. A61K 31/4353; A61P 35/00

(52) U.S. Cl. 514/283

(57) ABSTRACT

Topoisomerase I inhibitors are administered for the treatment of proliferative diseases according to a once every three day fixed dosing regimens. Furthermore, the invention provides the use of topoisomerase inhibitors to manufacture a medicament which can be used in a fixed dose treatment regimen.

This invention relates to the use of an improved regimen for the administration of topoisomerase I inhibitors, specifically gimatecan, for the treatment of patients suffering from proliferative diseases. According to the inventive regimen a topoisomerase I inhibitor can be administered as a fixed dose, once every three days, which tailors gimatecan's long terminal half-life with the aim to maintaining efficacy and with the potential to reduce toxicity as a result of drug accumulation compared to frequent once daily dosing. Alternatively, the present invention embraces a treatment regimen wherein a topoisomerase I inhibitor is administered at a dose in the range from 0.5 mg to 2.0 mg once every three days. The inventive dosing regimens provides an adequate level of drug exposure, for example above about 200-400 ng*hr/ml, which is below the range where unwanted side effect might occur. This regimen is to be administered continuously so that tumor cells are under a relatively constant drug exposure.

Camptothecin derivatives that are selective inhibitors of topoisomerase I, pharmaceutical formulations thereof and methods of making such compounds are described, for example, in U.S. Pat. No. 6,242,457, which is here incorporated by reference. Such compounds have been reported to have antitumor activity and are effective in treating proliferative diseases. In the inventive dosage regimen, the camptothecin derivative is 7-(t-butoxy)imino methyl camptothecin, or gimatecan.

The conventional approach to determine the dose for an anticancer agent is based on body surface area and finding the maximum tolerated dose (MTD). Fix dosing approach is used because gimatecan clearance is not predicted by body surface area as determined in 78 patients receiving different schedules of gimatecan in the completed phase I trial. Also, because of its half-life, daily dosing of the camptothecian derivative gimatecan causes toxic levels of the drug to accumulate in the body. It has been found that a fixed dose determined from the putative drug exposure range may provide a safer and more effective dose than doses determined from the putative drug exposure range rather than by the conventional MTD approach. In addition, the fix dosing approach reduces administration related errors and provides patients with ease in drug administration process. The rationale behind this dosing strategy employs the anti-angiogenic effect of this drug, the drug's cytotoxic mechanisms of action while tailoring gimatecan's clinical pharmacology profile (e.g., long terminal half-life). Due to the reduced toxicity, administration of gimatecan according to the present dosing schedule allows for improved combination therapy with other anticancer agents.

The optimal dose and schedule of gimatecan stems mainly from the putative therapeutic window identified from the PK/PD analysis of completed phase I data. The target range of cumulative gimatecan cycle exposure (AUC 5,000-10,000 (ng)(hr)/mL) was selected because it produces partial responses and yet is below the exposure where grade III/IV neutropenia and thrombocytopenia adverse events became evident (FIG. 1). Utilizing a daily dosing schedule, the projected daily upper limit of exposure will roughly yield AUC=2,000 (ng)(hr)/mL for D5, AUC=1,000 (ng)(hr)/mL for D10, and 666 (ng)(hr)/mL for D15 all of which exceed by 1.5 to 5 times the putative effective daily exposure identified (AUC=200-400 (ng)(hr)/mL) (FIG. 2) also known as the therapeutic window. This may explain why toxicity could not be differentiated among the three schedules; all three schedules exceeded the putative effective daily exposure and were in a potentially toxic range. This suggests that the optimal dose and schedule should employ a minimal effective dose which targets a daily exposure to no greater than 400 (ng)(hr)/mL with the aim of continual drug exposure, while taking into consideration the 86 hour terminal half-life of gimatecan. The flat dose is dosed every third day, or once every three days, or every 72 hours, or at one half-life to avoid potential accumulation. The starting doses are chosen to target a systemic exposure of 200-400 (ng)(hr)/mL (the “therapeutic window”) once every three days.

A fixed dose is preferred because the rational supporting dosing the patient according to body surface area does not stand in the case of the topoisomerase I inhibitor gimatecan and a fixed dose allows for ease of administration and reduced administration related errors which can arise when the dose must be calculated and rounded.

It is highly desirable to provide a dosing regime for a cytotoxic agent such as a topoisomerase I inhibitor, in a fixed dose; preferably an oral dose. It has also been found in accordance with the present invention that a cytotoxic agent such as a topoisomerase I inhibitor can be administered in a fixed dose on a every 3 day schedule, continuously without cycle differentiation.

### SUMMARY OF THE INVENTION

Thus, the present invention relates to a method of administering camptothecin derivatives such as gimatecan to a patient, which comprises administering a fixed dose of a pharmaceutically effective amount of the camptothecin derivative, or a pharmaceutically acceptable salt thereof, to the patient on a once every three days schedule, continuously without cycle differentiation. The doses is determined based on the therapeutic window, not the maximum tolerated dose (MTD).

### DESCRIPTION OF THE DRAWINGS

FIG. 1 shows that the gimatecan putative effective daily exposure is between 200 and 400 (ng)(hr)/mL.

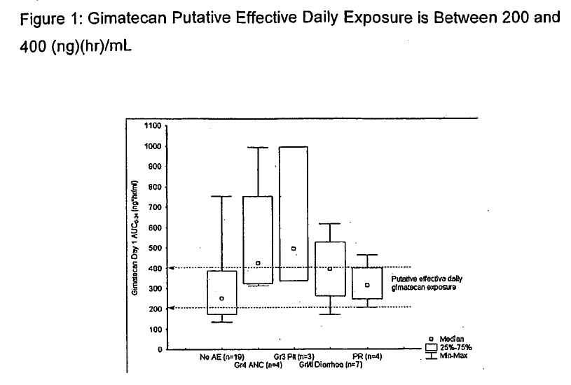

FIG. 2 shown that the gimatecan putative therapeutic window as defined by cumulative exposure is between 5,000 and 10,000 (ng)(hr)/mL.

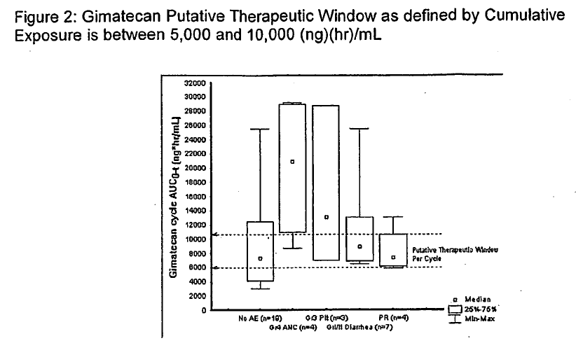

FIG. 3 shows the disposition of gimatecan using four different dosing schedules. The gimatecan concentration is compared to time.

FIG. 4 shows the lack of correlation between body surface area and gimatecan oral clearance. Gimatecan oral clearance is compared to body surface area (BSA).

### DETAILED DESCRIPTION OF THE INVENTION

The present invention relates to a method of administering a camptothecin derivative to a patient, which comprises administering a fixed dose of a pharmaceutically effective amount of the camptothecin derivative, or a pharmaceutically acceptable salt thereof, to the patient on a once every three days (or once every 72 hours) schedule, continuously without cycle differentiation.

Alternatively, the present invention relates to a method of administering a camptothecin derivative to a patient, which comprises administering the camptothecin derivative, or a pharmaceutically acceptable salt thereof, to the patient on a schedule at a fixed dose in the range from 0.5 mg to 2.0 mg once every three days, particularly a dose of 0.5 mg to 1.5 mg once every three days, continuously without cycle differentiation. In preferred embodiments the dose can be 0.5, 1.0, 1.5 or 2 mg every three days.

In a further embodiment, the present invention relates to a method of administering a camptothecin derivative to a patient, which comprises administering the compound, or a pharmaceutically acceptable salt thereof, to the patient on a schedule at a fixed dose in the range from 0.5 mg to 2.0 mg once every three days, continuously. The dose will be administered as a fixed dose either using a loading and maintenance approach or evenly distributed dose every 72 hours or once every three days that the total dose administered provides a maximum systemic exposure of 400 (ng)(hr)/mL.

A dose administered once every three days includes a dose administered once every 72 hours for example on Monday and Thursday then repeated the following week. For patient compliance, a placebo may be taken on the remaining days. For example for a Monday/Thursday dosing schedule as described above, placebo can be taken on the remaining days, i.e. Tuesday, Wednesday, Friday, Saturday and Sunday.

The invention further relates to a method of treating a proliferative disease in a patient, which comprises administering a pharmaceutically effective amount of a camptothecin derivative, or a pharmaceutically acceptable salt thereof, to the patient on a once every three day schedule continuously without dose differentiation.

Furthermore, the invention provides the use of a camptothecin derivative, for the manufacture of a medicament to be used for treating a proliferative disease, designed so that camptothecin is administered in a fixed dose once every three days.

In addition, the invention provides the according to the above, wherein the daily dose is from 0.5 mg to 2.0 mg.

In addition, the invention provides the use according to the description above, wherein the fixed dose is administered using a loading and maintenance approach or evenly distributed dose every 72 hours.

The invention also provides the use according to the description above, in which the camptothecin derivative is gimatecan.

Furthermore, the invention provides the use according to any the description above, wherein the proliferative disease is a solid tumor cancer.

The invention also provides the use according to the description above, wherein the daily dose is 0.5 mg.

In addition, the invention also provides the use according to the above description, wherein the fixed dose will provide a daily systemic exposure of AUC=200-400(ng)(hr)/mL).

The term “proliferative disease” especially includes solid tumors and refractory solid tumors.
Gimatecan is especially useful for inhibiting metastatic growth of a cancer. Thus, the present invention further relates to method of inhibiting metastatic growth in a patient with a cancer, which comprises administering a fixed dose of a pharmaceutically effective amount of gimatecan or a pharmaceutically acceptable salt thereof, to the patient on a once every three day schedule. Alternatively, the present invention further relates to method of inhibiting metastatic growth in a patient with a cancer, which comprises administering a pharmaceutically effective amount of gimatecan or a pharmaceutically acceptable salt thereof, to the patient on a once every three day schedule at a dose in the range from 0.5 mg to 2.0 mg once every three days, particularly a dose of 0.5 mg to 1.5 mg once every three days.

The invention also provides the use according to the description above, wherein the disease is a metastatic tumor.

According to one aspect of the present invention, gimatecan is given once every three days on a continuous basis, alone or in combinations with other cytotoxic drugs, or during and subsequent to other therapies, for example in chemotherapy. An oral administrated n of a fixed amount in the range from 0.5 mg to 2.0 mg every three days is contemplated as a pharmaceutically effective amount in the once every three day regimen. The administered dose may be administered in one dosage strength i.e. a single capsule or tablet, or as separate dosage strengths, i.e. two or more separate capsules or tablets, so that the total dose administered provides a maximum systemic exposure of 400 (ng)(hr)/mL.

Gimatecan is administered alone, or in combination with other therapeutic agents, for example chemotherapy. As a combination therapy, it is administered once every three days as described herein and any other therapeutic agent or agents are administered according to its established administration regimen.

### EXAMPLE 1

Patients with refractory solid tumors, enrolled into a phase I dose-finding study, receive a fixed gimatecan dose ranging from 0.5 to 2 mg yielding a systemic exposure of no more than 400 as determined from analysis of the completed phase I PK/PD data. Patients receive this fixed dose either using a loading and maintenance approach or an evenly distributed dose every 72 hours. Safety and tolerability of gimatecan, interpatient variability, correlation of exposure with plasma circulating biomarkers, and/or to clinical responses (efficacy and toxicity) are measured.

### EXAMPLE 2

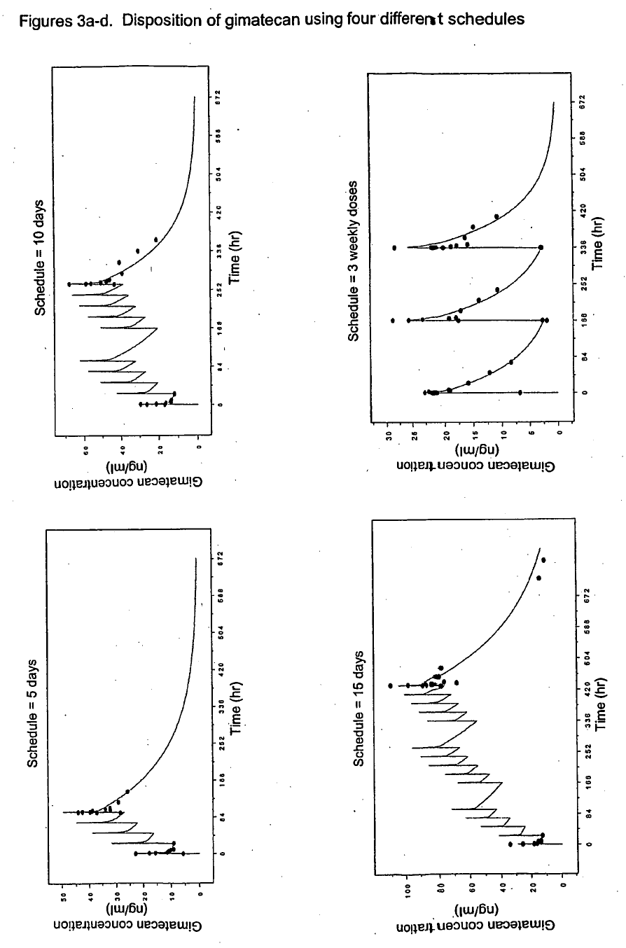

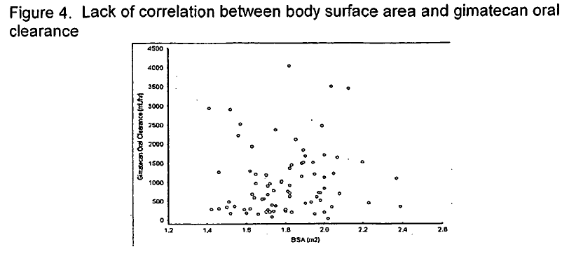

The disposition of oral gimatecan is evaluated in patients with refractory malignancies administered on a daily schedule for 5, 10, or 15 consecutive days or a weekly schedule for 3 weeks of a 28 day cycle. The disposition of gimatecan using these four schedules are depicted in FIGS. 3 (a-d). Clinical pharmacokinetics is assessed on the first and last days of the first cycle of daily dosing. Gimatecan has a long terminal phase, as the terminal harmonic mean half-life is estimated to be 86 hours from 10 patients receiving the weekly schedule. Drug accumulation is observed in both the weekly and daily schedule, which is consistent with the long terminal half-life of this drug. Gimatecan's apparent oral clearance is independent of dose suggesting linear kinetics and body surface area is not a good predictor of gimatecan clearance in 78 patients receiving four schedules (FIG. 4). However, patients were dosed based on their body surface area. Patients who responded to gimatecan had taken 0.45 mg, 0.9 mg, 1.1 mg or 1.6 mg which made it difficult to pick an effective dose.

1-8. (canceled)

9. A method of administering a camptothecin derivative to a patient, which comprises administering a pharmaceutically effective amount of the camptothecin derivative to the patient in a fixed dose once every three days.

10. A method of claim 9 wherein a daily dose of from 0.5 mg to 2.0 mg once every three days of the camptothecin derivative is administered to the patient.

11. A method according to claim 9 wherein the fixed dose is administered using a loading and maintenance approach or evenly distributed dose every 72 hours.

12. A method according to claim 9 wherein the camptothecin derivative is gimatecan.

13. A method of treating a proliferative disease in a patient which comprises administering a pharmaceutically effective amount of a camptothecin derivative to the patient on a once every three day schedule.

14. A method of claim 13 wherein the proliferative disease is a solid tumor cancer.

15. A method of claim 14 wherein a fixed dose of from 0.5 mg-2.0 mg once every three days of the camptothecin derivative is administered to the patient.

16. A method according to claim 15 wherein the fixed dose is administered using a loading and maintenance approach or evenly distributed dose every 72 hours.

17. A method of treating a solid tumor or a refractory solid tumor in a patient, which comprises administering a pharmaceutically effective amount of a camptothecin derivative at a fixed dose in the range from 0.5 to 2.0 mg once every three days.

18. A method of administering a camptothecin derivative to a patient, which comprises administering the camptothecin derivative to the patient on a once every three days schedule at a fixed dose in the range from 0.5 mg to 2.0 mg.

19. A method of claim 18 wherein the once daily dose is 0.5 mg.

20. A method of administering a camptothecin derivative to a patient by administering a fixed dose which provides daily systemic exposure of AUC=200-400 (ng)(hr)/mL).

21. A method of treating a proliferative disease comprising administering a camptothecin derivative in a fixed dose which provides daily systemic exposure of AUC between 200-400 (ng)(hr)/mL).
